(19) United States
(12) Patent Application Publication
(10) Pub. No.: US 2022/0357310 A1
(43) Pub. Date: Nov. 10, 2022

(54) FLATULENCE DETECTION DEVICE

(71) Applicants: Scott Hadley, Spokane, WA (US); Aaron Hays, Whittier, CA (US)

(72) Inventors: Scott Hadley, Spokane, WA (US); Aaron Hays, Whittier, CA (US)

(21) Appl. No.: 17/737,056

(22) Filed: May 5, 2022

Related U.S. Application Data
(60) Provisional application No. 63/184,775, filed on May 5, 2021.

(51) Int. Cl. G01N 33/497

(52) U.S. Cl. CPC G01N 33/497; G01N 2033/4975; G08B 5/36

(57) ABSTRACT

Disclosed are implementations of a detection device configured to detect flatulence. In particular, it is envisioned that the detection device will be used to detect patient flatulence after gastrointestinal surgery. In this way, healthcare providers can be certain the GI tract is properly functioning prior to patient discharge. An example detection device comprises: a control unit that includes a visual display; a gas sensor configured to detect at least one volatile organize compound found in flatulence; and a cable extending between the control unit and the gas sensor that is configured to electrically connect the gas sensor to the control unit. The cable is also configured to allow the gas sensor to be positioned near the anus of a patient; the visual display of the control unit is configured to provide a visual indicator when a volatile organize compound found in flatulence is detected by the gas sensor.

### CROSS REFERENCE TO RELATED APPLICATION

This application claims the benefit of U.S. Provisional Application Ser. No. 63/184,775, filed on May 5, 2021, the entirety of which is incorporated herein by reference.

### TECHNICAL FIELD

This disclosure relates to implementations of a flatulence detection device.

### BACKGROUND

After gastrointestinal surgery, a patient is not allowed to eat until they have a bowel movement or expel flatus through the anus, commonly referred to as “passing gas”. A bowel movement or flatulence serve as a clear sign the gastrointestinal tract is functioning and that postoperative ileus, defined as impairment of gastrointestinal (GI) motility after intra-abdominal or nonabdominal surgery, is not a concern.

What is needed is a device capable of detecting flatus expelled from the anus of a patient. Optimally, such a device would include a sensor that can be positioned adjacent the anus. This would reduce, and potentially eliminate, the sensor detecting flatus expelled by someone other than the patient.

Accordingly, it can be seen that needs exist for the flatulence detection device disclosed herein. It is to the provision of a flatulence detection device configured to address these needs, and others, that the present invention is primarily directed.

### SUMMARY OF THE INVENTION

It is to be understood that this summary is not an extensive overview of the disclosure. This summary is exemplary and not restrictive, and it is intended neither to identify key or critical elements of the disclosure nor delineate the scope thereof. The sole purpose of this summary is to explain and exemplify certain concepts of the disclosure as an introduction to the following complete and extensive detailed description.

Disclosed are implementations of a detection device configured to detect flatus expelled through the anus. In particular, it is envisioned that the detection device will be used to detect patient flatulence after gastrointestinal surgery. In this way, healthcare providers can be certain the GI tract is properly functioning prior to patient discharge.

An example detection device comprises: a control unit that includes a visual display; a gas sensor configured to detect at least one volatile organize compound found in flatulence; and a cable extending between the control unit and the gas sensor that is configured to electrically connect the gas sensor to the control unit. The cable is also configured to allow the gas sensor to be positioned near the anus of a patient; the visual display of the control unit is configured to provide a visual indicator when a volatile organize compound found in flatulence is detected by the gas sensor.

An example method of using a detection device comprises: providing a detection device configured to detect flatulence, the detection device comprising: a control unit that includes a visual display; a gas sensor configured to detect at least one volatile organize compound found in flatulence; and a cable extending between the control unit and the gas sensor that is configured to electrically connect the gas sensor to the control unit. The cable is also configured to allow the gas sensor to be remotely positioned relative to the control unit; the visual display of the control unit is configured to provide a visual indicator when a volatile organize compound found in flatulence is detected by the gas sensor. The method also includes the step of positioning the gas sensor near the anus of the patient.

### BRIEF DESCRIPTION OF THE DRAWINGS

FIG. 1 illustrates a perspective view of a flatulence detection device according to the principles of the present disclosure. The cable of the flatulence detection device is shown with a symbolic break in its length.

FIG. 2 illustrates a perspective view of the control box shown in FIG. 1, wherein an LED of the visual display is illuminated.

FIG. 3 illustrates another perspective view of the control box shown in FIG. 2, wherein two LEDs of the visual display are illuminated.

FIG. 4 illustrates a perspective view of the example gas sensor.

FIG. 5 illustrates an diagram of an example electronic circuit for the flatulence detection device shown in FIG. 1.

Like reference numerals refer to corresponding parts throughout the several views of the drawings.

### DETAILED DESCRIPTION

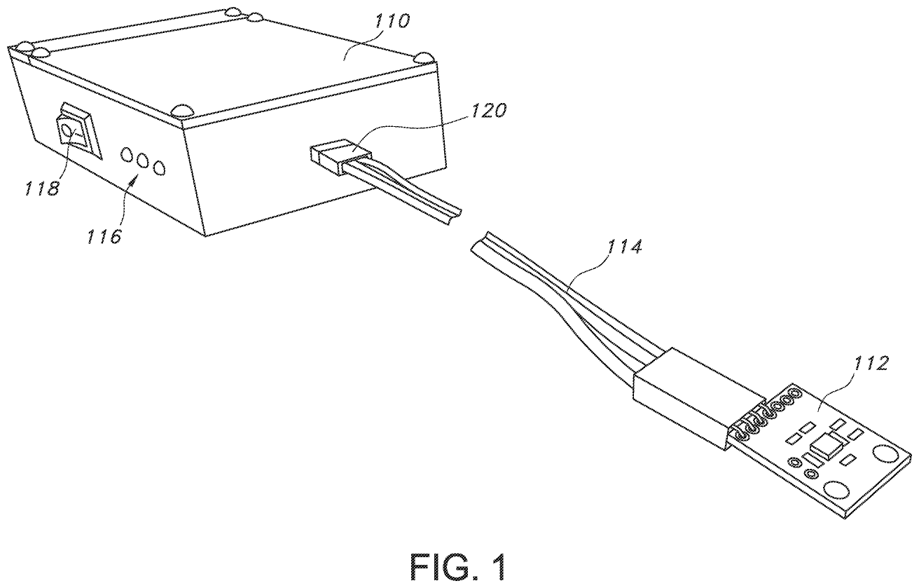

FIG. 1 illustrates an example flatulence detection device 100 according to the principles of the present disclosure. The flatulence detection device 100 is configured to detect flatus expelled through the anus. In particular, it is envisioned that the detection device 100 will be used to detect patient flatulence after gastrointestinal, or other, surgery. In this way, healthcare providers can be certain the GI tract is properly functioning prior to patient discharge.

As shown best in FIG. 1, the flatulence detection device 100 comprises a control unit 110 connected to a gas sensor 112 by a cable 114. The gas sensor 112 detects methane, and other volatile organic compounds (VOCs) found in flatulence. A visual indication that flatulence has occurred is generated by a visual display 116 when flatulence is detected.

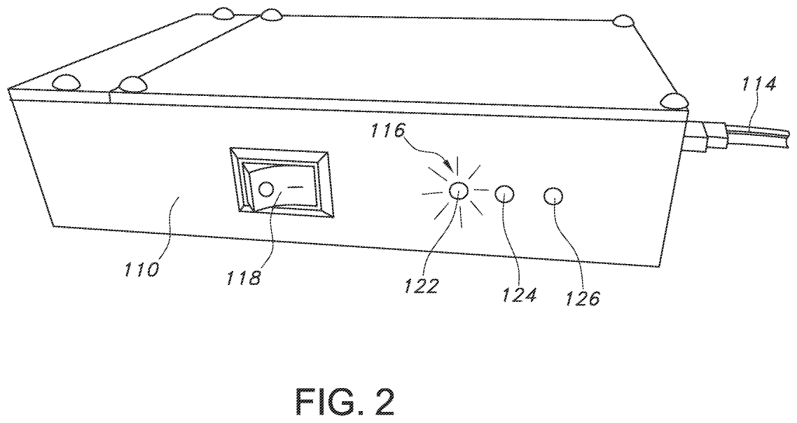

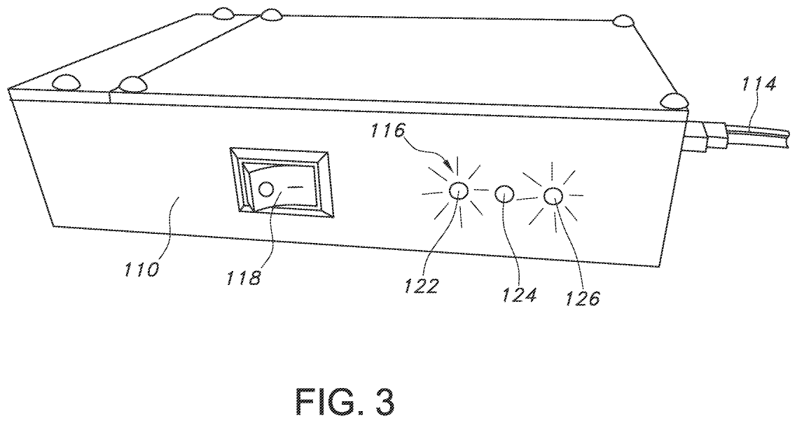

As shown best in FIGS. 2 and 3, the visual display 116, comprised of three light-emitting diodes (LEDs), is located on the exterior of the control unit 110. The control unit 110 also includes an ON/OFF switch 118 and a socket 120 configured to interface with an end of the cable 114. Power for the control unit 110, and the flatulence detection device 100 as a whole, is provided by one or more batteries contained within the control unit 110. Though, in some implementations, a power cord attached to an external power supply, such as a wall outlet, could be used to power the flatulence detection device 100.

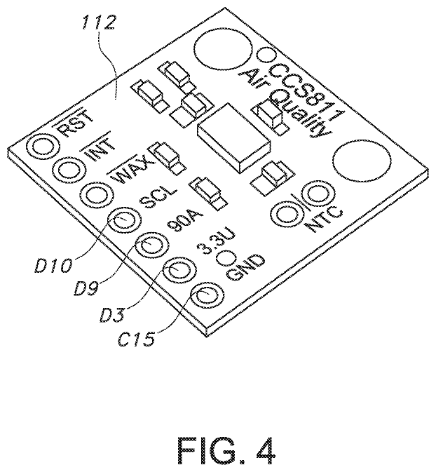

The gas sensor 112 of the flatulence detection device 100 is configured to detect methane, and other VOCs found in flatus. The gas sensor 112 shown in FIGS. 1 and 4 is a CCS811 Air Quality Breakout sold by SparkFun Electronics®. However, it should be understood that other gas sensors capable of detecting VOCs, such as methane, found in flatulence can be used. The gas sensor 112 is removably connected to one end of the cable 114.

The cable 114 allows the gas sensor 112 to be remotely positioned relative to the control unit 110 of the flatulence detection device 100. The cable 114 also electrically connects the gas sensor 112 to the control unit 110 of the flatulence detection device 100. In this way, the gas sensor 112 is placed in circuit with the visual display 116, the ON/OFF switch 118, and other electrical components of the control unit 110. The cable 114 has a length sufficient to facilitate the gas sensor 112 being positioned near, or adjacent, the anus of a patient.

The cable 114 is configured to be detached from the socket 120 of the control unit 110 by a user. In this way, the gas sensor 112 and cable 114 can be replaced between uses. This prevents the flatulence detection device 100 from becoming a vector for harmful pathogens.

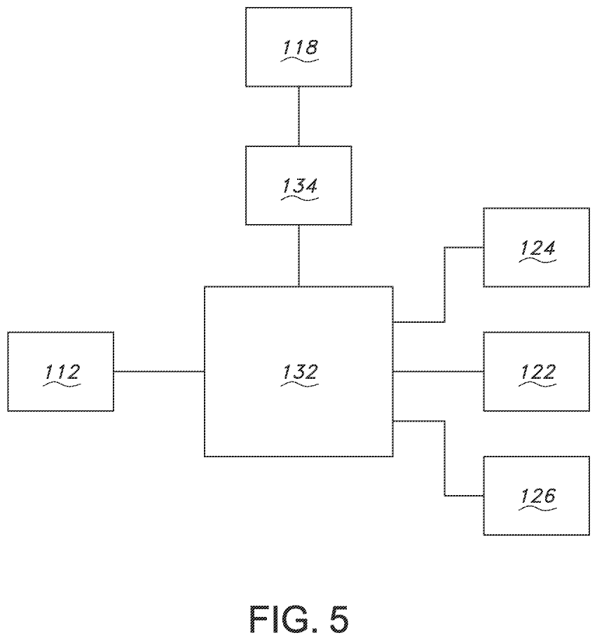

FIG. 5 illustrates an example electronic circuit 128 for the flatulence detection device 100 shown in FIG. 1. The example electronic circuit 128 has been removed from the housing 130 of the control unit 110 and includes the gas sensor 112, the cable 114, the LEDs (122, 124, 126) of the visual display 116, the ON/OFF switch 118, and additional components (e.g., a microcontroller 132 and power source 134) necessary to facilitate the function of the flatulence detection device 100 described herein.

The microcontroller 132 (or “MCU”) includes one or more CPUs, nonvolatile memory, and programable input/output peripherals mounted on a printed circuit board (PCB). The MCU 132 is configured to enable the flatulence detection device 100 to function as described herein.

The flatulence detection device 100 is turned ON using the ON/OFF switch 118 on the control unit 118. This energizes the electronic circuit 128 of the detection device 100, causing a first LED 122 of the visual display 116 to illuminate. If the battery is low, a second LED 124 of the visual display 116 illuminates. If methane or other VOCs found in flatus are detected by the gas sensor 112, a third LED 126 of the visual display 116 illuminates. In this way, a visual indication that flatulence has occurred is provided to anyone observing the visual display 126 of the control unit 110. Toggling the ON/OFF switch 118 OFF then back ON will reset the flatulence detection device 100.

When using the flatulence detection device 100, the gas sensor 112 is positioned near, or adjacent, the anus of a patient. In this way, flatus exiting the anus of the patient can be detected. This positioning of the gas sensor 112 may help to ensure that flatus originating from someone other than the patient is not detected and reported by the flatulence detection device 100. Medical tape, or a suitable adhesive, can be used to position the gas sensor 112 on the patient. Alternatively, the gas sensor 112 could be incorporated into an undergarment worn by the patient. For example, such an undergarment may include an opening, or pocket, for the gas sensor 112.

Although not shown in the drawings, it will be understood that suitable wiring and traces connect the electrical components of the flatulence detection device 100 disclosed herein.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A detection device configured to detect flatulence, the detection device comprising:
a control unit that includes a visual display;
a gas sensor configured to detect at least one volatile organize compound found in flatulence; and
a cable extending between the control unit and the gas sensor that is configured to electrically connect the gas sensor to the control unit;
wherein:
the cable is also configured to allow the gas sensor to be positioned near the anus of a patient; and
the visual display of the control unit is configured to provide a visual indicator when a volatile organize compound found in flatulence is detected by the gas sensor.

2. The detection device of claim 1, wherein the gas sensor is configured to detect methane.

3. The detection device of claim 2, wherein the visual indicator is an illuminated LED.

4. A method of detecting flatus exiting the anus of a patient, the method comprising:
providing a detection device configured to detect flatulence, the detection device comprising: a control unit that includes a visual display; a gas sensor configured to detect at least one volatile organize compound found in flatulence; and a cable extending between the control unit and the gas sensor that is configured to electrically connect the gas sensor to the control unit; wherein: the cable is also configured to allow the gas sensor to be remotely positioned relative to the control unit; and the visual display of the control unit is configured to provide a visual indicator when a volatile organize compound found in flatulence is detected by the gas sensor; and
positioning the gas sensor near the anus of the patient.

5. The method of claim 4, wherein the gas sensor is configured to detect methane.

6. The method of claim 5, wherein the visual indicator is an illuminated LED.
